(19) United States
(12) Patent Application Publication
(10) Pub. No.: US 2022/0023661 A1
(43) Pub. Date: Jan. 27, 2022

(54) DEVICE AND PROCEDURES TO ENHANCE INFRARED RADIATION ABSORPTION OF TISSUE

(71) Applicant: Sang Beom Lee, Harrison City, PA (US)

(72) Inventor: Sang Beom Lee, Harrison City, PA (US)

(21) Appl. No.: 17/385,233

(22) Filed: Jul. 26, 2021

Related U.S. Application Data
(60) Provisional application No. 63/056,133, filed on Jul. 24, 2020.

(51) Int. Cl. A61N 5/06; A61K 45/06

(52) U.S. Cl. CPC A61N 5/0625; A61N 5/0624; A61N 2005/0637; A61N 2005/0659; A61N 2005/0626; A61K 45/06

(57) ABSTRACT

A method of treating a patient via infrared radiant heating includes increasing the radiant energy per unit time of infrared radiation applied to the patient's skin over a duration of a treatment protocol.

### CROSS-REFERENCE TO RELATED APPLICATIONS

This application claims benefit of U.S. Provisional Patent Application Ser. No. 63/056,133, filed Jul. 24, 2020, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Infrared (IR) electric radiant heating uses electromagnetic radiation with wavelengths between 760 nm and 100 μm. Short or near infrared waves range from 760 nm to 1.4 μm. Emitters in those ranges are also referenced as bright because some visible light is emitted. Medium infrared emitters range between 1.4 μm and 3 μm, and far infrared or dark emitters exceed 3 μm. IR electric radiant heaters use heating elements that reach high temperatures. An IR heater can be used as a deliberate heating source. The IR element is usually packaged inside a glass envelope which may, for example, resemble a light bulb or is wrapped around a ceramic column with a reflector to direct the energy output away from the body of the heater.

The IR element emits infrared radiation that travels through air or space until it hits an absorbing surface where it is partially converted to heat and partially reflected. That portion converted to heat directly warms people and objects in the room, rather than warming the air. This type of IR heater is particularly useful in areas of uneven air flows. For example, such IR heaters may be used in infrared saunas to heat the occupants.

When radiation from an IR heater falls on any surface, the radiation is absorbed by the molecules on the surfaces and the molecules begin oscillating or vibrating. With continued IR radiation, those molecules continue to absorb energy and their frequency of oscillation increases. In addition to the dangers of touching the hot IR source or element, high-intensity, short-wave infrared radiation may cause indirect thermal burns when the skin is exposed for too long or the heater is positioned too close to the subject.

Maximum exposure is recommended for optimal treatment without bums. While the distance should be adjusted for personal comfort, in general, it is recommended that the lamp should never be placed closer than 18 inches to the surface toward which it is directed. Once again, IR radiation can cause burns if improperly applied. It should not be used near an infant or a sleeping or unconscious person. An electrically powered IR radiant heater should also not be used by people with sensitive skin or poor blood circulation. The skin and eyes are especially vulnerable to the potential hazards of this radiation. Because of the intensity of radiant heat emitted, skin can bum rather quickly. Overheating or burning is identifiable through the appearance of a violet-colored skin reaction. There is a critical need to maximize the radiation at the body skin but, at the same time, avoid burn injuries. In addition, a method for lowering perceived temperature is essential for a long-term radiation exposure that can range in time from 20 minutes to several hours.

Increasing evidence suggests that IR can carry out photostimulation and photobiomodulation effects that particularly benefit, for example, neural stimulation, wound healing, and cancer treatment. A better understanding of new developments and biological implications of IR could help to improve therapeutic effectiveness or aid in development of medical treatments using IR electric radiant heating. IR therapy is relatively new to the skincare industry. Many factors influence the therapeutic effects of IR radiant heating, including fluence, irradiance, treatment timing and repetition, pulsing, and wavelength.

Compared to other ranges of energy, IR penetrates the skin on a deeper level. Moreover, IR irradiation is a non-invasive treatment. Red light therapy reaches the body 8-10 millimeters below the surface of the skin, effecting blood vessels, lymph pathways, nerves, and hair follicles. IR therapy is FDA approved for chronic joint pain, wound healing capabilities, wrinkles, hair loss, and acne.

IR radiant systems are disclosed in, for example, U.S. Pat. Nos. 4,186,294, 6,084,242, 6,443,915, 6,673,096, 6,719,780, 7,503,926, 7,693,580, 7,783,361, 7,785,35B, 8,192,473, and 8,252,033. A number of disclose therapeutic IR radiant systems set forth designs of apparatuses, specific wavelengths to be used, and areas of skin to be radiated. Despite being well-documented in the design and operation, they lack treatment procedures to optimize or maximize therapeutic effects. Therefore, there is a critical need for a new IR radiant system that can provide well-organized and controlled treatment procedures and algorithms to increase the total fluence of absorbed radiation into skin. In addition, a number of currently available IR radian systems use several small-diameter IR heaters that do not provide enough radiation to patients and create untreated skin areas. An improved interface with multiple controllers and monitors between patients and medical practitioners is also necessary for a long-term treatment (for example, from 20 minutes to several hours).

### SUMMARY

In one aspect, a method of treating patient via infrared radiant heating includes increasing the radiant energy per unit time of infrared radiation applied to the patient's skin over a duration of a treatment protocol. The radiant energy per unit time may, for example, be increased in a manner that the patient, after an initial warming period, does not perceive a significant temperature increase over the duration of the treatment protocol. In a number of embodiments, the infrared radiation in a range of wavelengths between 760 nm and 100 μm is applied to the patient's skin.

In a number of embodiments, the infrared radiation is applied using a source comprising one or more large reflectors/sources. Each reflector may, for example, have a diameter between 5 inches and 36 inches. Tach reflector may, for example, include a beam guard ring to narrow the beam of radiation. The materials for the beam guard ring may, for example, include a transparent material or include a non-transparent metal. In a number of embodiments, the beam guard is constructed from a transparent polymer. The transparent polymer may, for example, be a polycarbonate or a polymethylmethacrylate. In the case that the metal is used, the metal may, for example, be aluminum, titanium, or stainless steel. In a number of embodiments, the radiant energy is applied using a single reflector/source. The infrared radiation may, for example, be applied via a device supported on caster wheels.

In a number of embodiments, the method further includes providing a patient interface to provide information to the patient. The method may, for example, further include providing a patient control mechanism via which the patient can stop the procedure. The patient may, for example, be provided a monitor which includes a mechanism to stop the procedure.

In a number of embodiments, the infrared radiation is changed over the duration of the treatment protocol in at least one of a linearly increasing manner, an incrementally increasing manner or an incrementally increasing manner in which the energy is pulsed.

In a number of embodiments, the method further includes providing at least one speaker to provide audible information to the patient or a medical practitioner.

The distance between a source of infrared radiation and the skin of the patient may be controlled to be within a predetermined range throughout the treatment protocol. The radiant energy per unit time of infrared radiation applied to the patient's skin may, for example, be increased to increasing plateaus during the treatment protocol. The duration of the treatment protocol may, for example, be between 20 minutes to 3 hours.

In a number of embodiments, the method further includes providing a mesh over clothing of the patient during the treatment protocol to control a gap between the patient's skin and the clothing. The mesh may, for example, include an elastomeric material.

The treatment protocol may, for example, be used to treat a condition other than a skin condition. In a number of embodiments, the treatment protocol is used to treat an infection. The treatment protocol may, for example, be used to treat a viral infection or a bacterial infection. The treatment protocol may, for example, be used to treat fever, chills, nausea, shortness of breath or coughing. In a number of embodiments, the treatment protocol is used to treat a Coronavirus disease, COVID-19, Severe acute respiratory syndrome (SARS), a common cold, an influenza, chickenpox, cold sores, rabies, Ebola, AIDS (HIV), or an avian influenza.

The patient may also be treated with at least one therapeutic agent which may be administered as known in the medical arts (for example, orally, intravenously, transdermally, etc.). The therapeutic agent may, for example, be an antibiotic, an antiviral, a pain killer, an anti-inflammatory or a health supplement such as a vitamin, a mineral, a fiber, a fatty acids or an amino acid.

The method may further includes providing a beverage comprising water to patient consumption during the treatment protocol. The patient may, for example, be instructed to consume the beverage periodically.

In another aspect, a system for treating patient includes a source for applying infrared energy to the patient for infrared radiant heating, and a control system in operative connection with the source, the control system being conjured to control the source to increase radiant energy per unit time of infrared radiation applied to the patient over a duration of a treatment protocol.

In a number of embodiments, the control system is configured so that the radiant energy per unit time is increased in a manner that the patient, after an initial warming period, does not perceive a significant temperature increase over the duration of the treatment protocol.

In a further aspect, a method of treating patient via infrared radiant heating includes providing a mesh other form-fitting garment/system through which infrared energy is readily transmissible over clothing of the patient during a treatment protocol to control a gap between the patient's skin and the clothing and applying infrared radiation to an area covered by the mesh or other garment/system. The mesh or other garment/system may, for example, include an elastomeric material.

In a number of embodiments, IR radiant heating devices, systems and methods hereof that can, for example, provide: i) well organized treatment procedures to maximize radiation therapy ranging to time from 20 minutes to several hours, ii) a deliberate heating to a desired location or area, using large one or two reflectors, iii) multiple interfaces and controllers between patients and medical practitioners for long-term radiation exposure, iv) use in connection with medications such as vitamins and pain killers to accelerate the therapeutic effects during the IR radiant treatment, and vi) a wearable mesh to minimize the gap between skin and clothing when a patient does not want to take off any article of clothing.

The devices, systems and methods hereof enhance, optimize or maximize radiation absorbed by a patient's skin through, for example, pulses or continuous infrared radiations to, for example, provide treatment of severe symptoms of viral and/or bacterial infections such as fever, chill, nausea, shortness of breath, and coughing. Treatment protocols enabled by the system hereof may, for example, be used to viral diseases such as coronavirus infections (including, for example, COVID-19, severe acute respiratory syndrome (SARS), and the common cold), various strains of influenza, chickenpox, cold sores, rabies, Ebola, AIDS (HIV).

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a schematic, side perspective view of an embodiment of an electrically powered IR radiant system hereof which includes an IR radiant filament, a reflector, swivel caster wheels, two monitors for communication with a patient and medical practitioner(s).

FIG. 2 illustrates an embodiment of a gradient heating procedure to minimize perceived temperature for patients and maximize total IR fluence absorbed in skin.

FIG. 3 illustrates an embodiment of an incremental heating procedure to minimize perceived temperature for patients and maximize total IR fluence absorbed in skin.

FIG. 4 illustrates an embodiment of a pulse heating procedure to minimize perceived temperature for patients and maximize total IR fluence absorbed in skin.

FIG. 5 illustrates the skin area that is radiated by a single large reflector heater, which, in a number of treatment modalities, may, for example, encompass cervical lymph nodes, two axillary lymph nodes, and two pelvic lymph nodes.

FIG. 6 illustrates an embodiment of a mesh (formed, for example, from an elastomeric material) which may be used to minimize a gap between skin and patient's clothing for better radiation transfer.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a reflector” includes a plurality of such reflectors and equivalents thereof known to those skilled in the art, and so forth, and reference to “the reflector” is a reference to one or more such reflectors and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”, “circuitry” or “circuit,” as used herein include, but are not limited to, hardware, firmware, software or combinations of each to perform a function(s) or an action(s). For example, based on a desired feature or need. a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as operational amplifiers, Digital to Analog Converters (DACs), Analog to Digital Converters (ADCs), Pulse Width Modulated (PWM) circuitry, wired serial communication (UART, SPI, USB) devices, radio frequency communication devices, random access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions. A controller may also contain analog, discrete component, and integrated circuit blocks such as amplifiers, differentiators, integrators, oscillators, control knobs, and display devices.

The term “logic,” as used herein includes, but is not limited to. hardware, firmware, software or combinations thereof to perform a function(s) or an action(s), or to cause a function or action from another element or component. Based on a certain application or need, logic may, for example, include a software controlled microprocess, discrete logic such as an application specific integrated circuit (ASIC), or other programmed logic device. Logic may also be fully embodied as software. As used herein, the term “logic” is considered synonymous with the term “circuit.”

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

In a number of embodiments, IR radiant heaters, IR heaters, or IR radiant heating systems hereof may, for example, include one or more IR filaments, reflectors that reflect IR radiation to the patient. IR reflectors hereof may be attached to adjustable beds or otherwise configured to maintain a controlled distance between the IR source and skin for the patient's position (for example, standing, sitting etc.). In a number of such embodiments, IR radiant systems hereof include an algorithm to enhance, optimize or maximize the total IR fluence (that is, the time integrated energy per unit area) that a patient can receive, and the system has monitors for both patients and medical practitioners.

The devices, systems and methods hereof have numerous applications including, but not limited to, mitigating infections (for example, various viral or bacterial infections), improve immunity, wound healing, and skin care.

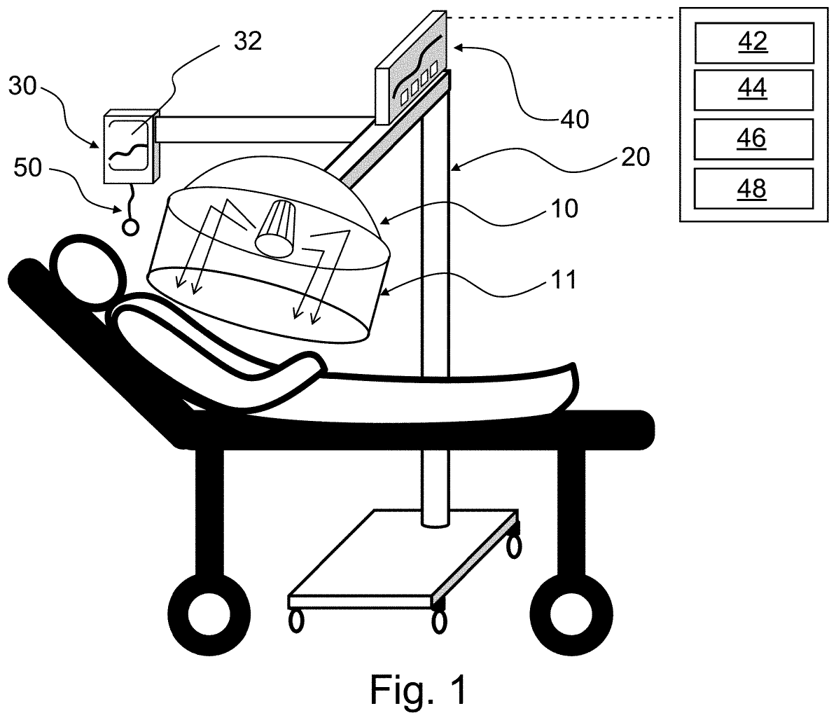

FIG. 1 illustrates an embodiment of an IR radiant system hereof that includes an IR source, an adjustable bed, and programmable monitors. Reflector (10) delivers IR radiation to patients in a controlled manner as described herein. In addition, reflector (10) can be situated in a way to avoid any radiation to the patient's eyes. Reflector (10) is mounted to a stand (20) in the illustrated embodiment. A safety ring (11) can be attached to the reflector (10). A beam guard ring (11) may be provided for a narrow beam of radiation. Beam guard ring (11) may, for example, be made of a polymer such as polycarbonate, plexiglass, etc. or a metal such as aluminum.

A patient interface system (30) may be provided including a monitor (32) for patients to view the status of the treatment and/or other interface devices to provide information to the patient. Patient interface (30) may also include an emergency shut-off switch (50) which may be wired or wireless for use by the patient in case of, for example, patient discomfort. A control system (40) in operative connection with the IR source may, for example, include circuitry for operation and control of the IR source. As illustrated schematically in FIG. 1, control system (40) may, for example, include a processing system (42),a memory system (44) in operative connection with processing system (42), an input/output system (46) and a user interface system (48) (including, for example, a touchpad, a touchscreen, a keyboard, a mouse, one or more speakers for communication of audible information, one or more monitors for communication of visual information, one or more devices for communication of tactile information, etc. as known in the computer arts) for use by medical practitioners who may, for example, be provided patient information and control parameters such as exposure time, temperature, and treatment procedures. Both patient interface system (30) and control system (40) may have speakers to inform patients and medical practitioners about the progress by providing information such as start, remaining time, the current treatment step, and finish.

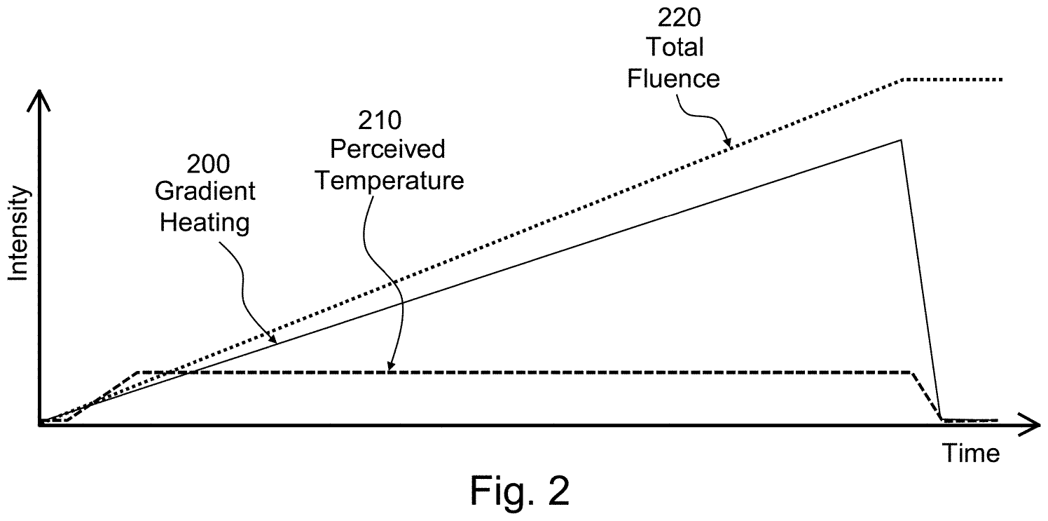

Software executable by processing system (42) may, for example, be stored in memory system (44) to control the IR source. Treatment protocols including, for example, a level of energy provide over treatment time may be selectable and/or programmable by a medical practitioner. FIG. 2, for example, shows an embodiment of a gradient radiation treatment protocol that enhances or maximizes the total fluence absorbed by patient skin (as, for example, compared to currently available IR radiation systems). Gradient heating treatment protocol 200 starts slowly. The patient will feel heat as the treatment progresses. However, after a certain IR exposure, the patient's perceived temperature 210 plateaus because patient's skin doesn't feel heat as the skin absorbs more fluence over time. Total fluence continue to increase and will become plateaued once the application of radiation stops.

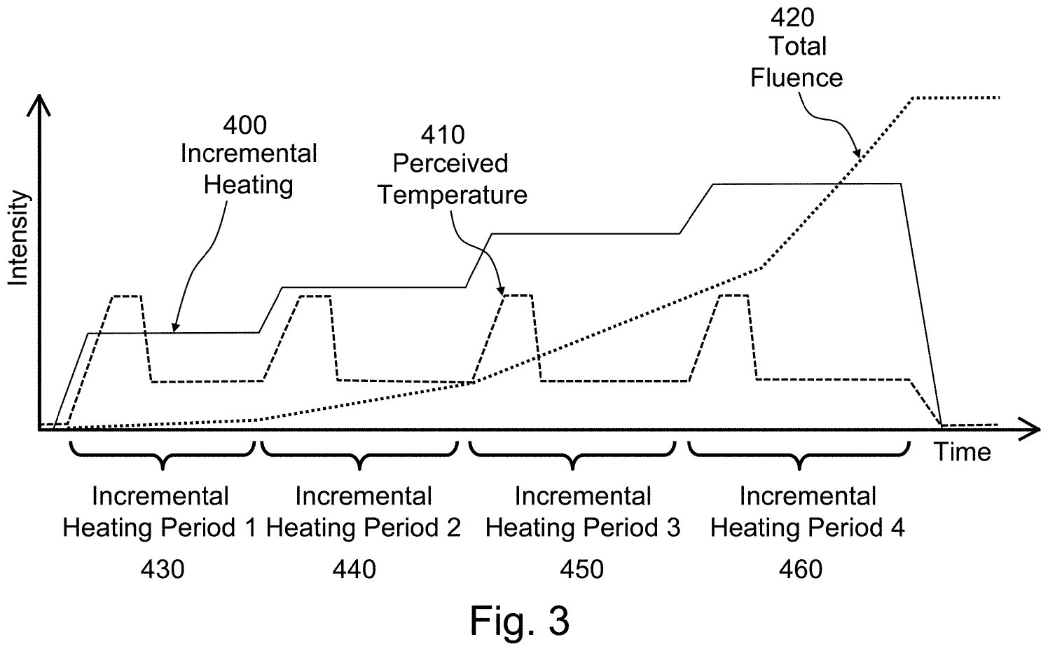

FIG. 3 illustrates an embodiment of an incremental radiation treatment protocol that enhances or maximizes the total fluence absorbed by patient skin. In general, the radiant energy per unit time applied to the patient is increased over at least a portion of the duration of the treatment procedure. Gradient heating treatment protocol 400 starts slowly and the patient will feel the heat. As described above, after a certain IR exposure, the patient's perceived temperature 410 plateaus and/or decreases because the skin the absorbed fluence increases over time. In the embodiment of FIG. 3, after a first incremental heating period 430, a second incremental heating period 440 starts with a stronger or higher level of IR heating. The patient will once again feel some heat (burst effect) but soon the patient loses the perception of heating. In general, perceived temperature 410 may be the same as the perceived temperature of the heating period 430 even though the total radiation absorbed over time (accumulated fluence) in the skin after the second incremental heating period 440 is increased. FIG. 2 shows only up to a fourth step (460) of increasing intensity, but medical practitioners may repeat such steps as many times as desired for a patient-dependent treatment protocol (for example, depending on the patient's health situations). By repeating treatment cycles, the patient can receive an enhanced, optimized or maximized accumulated fluence without feeling overheated. The skin can adapt to the temperature change between each step without damage to the skin.

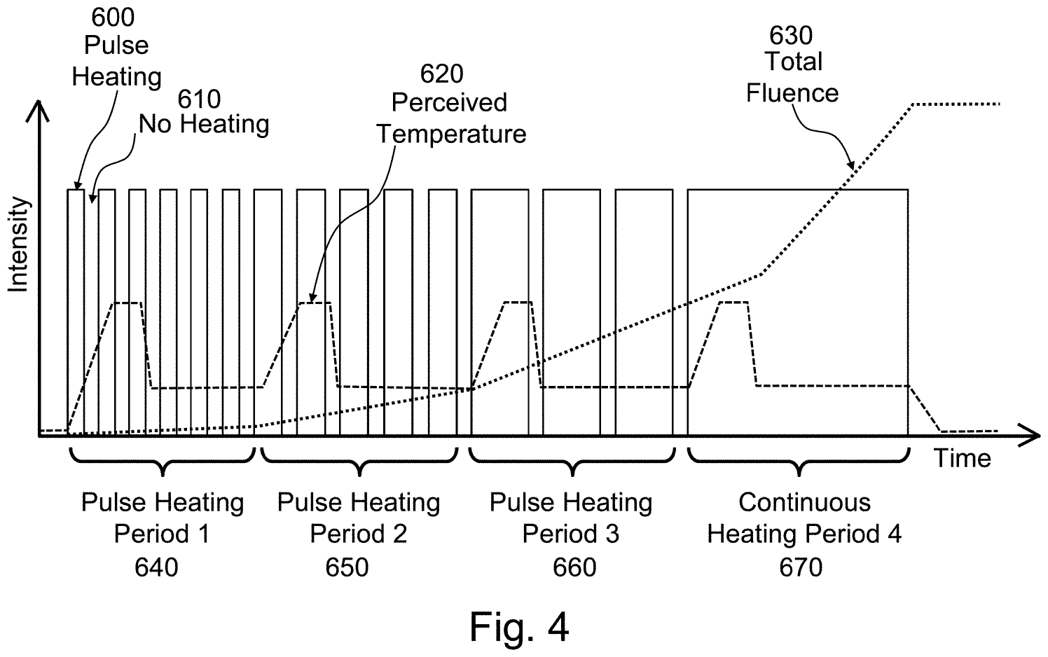

In addition to the continuous incremental treatment procedures described above, IR radiation treatments may be performed using pulses of radiation. FIG. 4 illustrates an embodiment of a pulse radiation treatment procedure or protocol that enhances, maximizes or optimizes the total fluence absorbed by a patient's skin. In the illustrated embodiment, a short-pulse, radiant heating treatment protocol 600 starts in pulse heating period 1 (640). The patient feels heat, but after a certain IR exposure, the patient's perceived temperature 620 decreases because patient's skin does not feel heat as the skin continues to absorb more fluence over time. Pulse radiation period 2 (650) starts with longer pulse radiant heating. The patient will feel some heat (burst effect), but soon will lose the feeling. Thus, perceived temperature 620 will be same as the perceived temperature in period 1 although the total radiation accumulated fluence in the skin in pulse heating period 2 (650) is much higher than in pulse radiation period 1 (640). Pulse radiation period 3 (660), in which the time period of each pulse is once again increased, delivers more energy to the skin than pulse period 2 (650) with similar perceived temperatures as experienced in pulse period 2 (650).

The treatment protocol (600) of FIG. 4 also includes a final stage which is a continuous (or non-pulsed) radiation period 4 (670). In that regard, further increasing the on-time period of each pulse may result in negligible gap (or off-times) between pulses. Thus, instead of a pulsed radiation treatment, the treatment may become a continuous radiation treatment in a later stage or stages. The continuous treatment of period 4 delivers the highest IR heating fluence but perceived temperature is similar to that in pulse radiation period 3 (660). FIG. 4 shows only three pulse radiation periods and one continuous radiation period, but a practitioner or person skilled in the art will realize that one can deliver fewer that or greater than 4 treatment periods, depending, for example, on the patient's skin and/or overall health conditions. By repeating pulse radiation and sequentially increasing pulse on-time in subsequent pulse radiation periods, the patient can receive an enhanced, optimized of maximized accumulated fluence, while not feeling overheated and, therefore, avoiding skin damage.

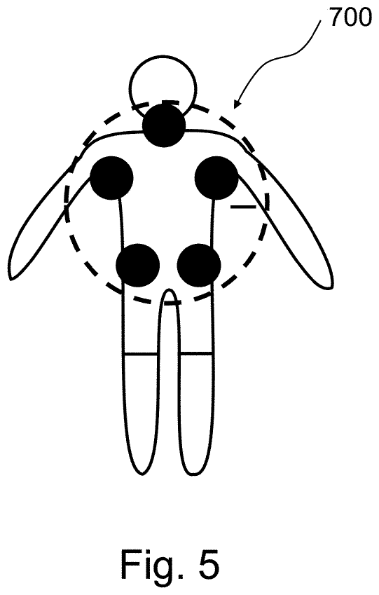

FIG. 5 illustrates the skin area that may, for example, be radiated by a single or a plurality of large reflector IR heater(s) in a number of embodiments hereof. In a number of treatment protocols, the skin area (700) may desirably include cervical lymph nodes (on neck), two axillary lymph nodes (under shoulders), and two pelvic lymph nodes. The entire skin area within the circle of FIG. 5 may, for example, be radiated evenly and concurrently to maximize the therapeutic effect.

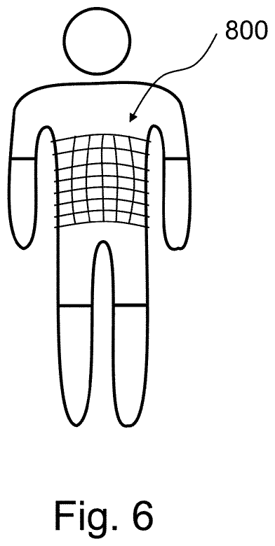

FIG. 6 illustrates a patient wearing an embodiment of a mesh system or garment (800) hereof during the IR treatment. Mesh garment (800) may, for example, be formed of an elastic material to facilitate conformance thereof to body shape. Mesh garment (800) may, for example, be used to reduce the gap between the patient's skin and the patient's clothing layer during treatment, thereby increasing the effectiveness of the radiant heating transfer to the skin.

Without limitation to any mechanism, the physiological response of the body whereby a patient acclimatizes to heat, and perceived temperature decreases, may, for example, be a form of “sensory adaptation” wherein, if a sensation doesn't change over time, the body begins to “tune it out.” The body adapt or get used to a particular imposed temperature condition by a process called thermoregulation in which the blood vessels near the skin vasodilate (open up valves allowing blood to be pumped nearer the surface of the skin). As the blood flows along the vessels it radiates heat away from your body (since the source of heat is warming the body) so that the body maintains a constant internal environment or homeostasis. Perceived temperature may not stay the same because the body is radiating just as much heat as it's conducting. For example, when immersed in hot water, the heat sensors at the surface of the skin don't notice a relative high temperature of the water as they're the same temperature.

In a number of embodiments hereof, the treatment room or at least the area surrounding the patient is kept relatively cool (for example, at 25° C. or below or at 20° C. or below. In a number of embodiments, the temperature is kept in the range of approximately 4 to 25° C. or between 4 and 20° C. In general, the cooler or colder the temperature can be maintained surrounding the patient during treatment (while accounting or providing for patient comfort), the better. The patient body will release heat and possible sweat to aid in heat transfer during an intense treatment. A cool or cold room will facility heat transfer from the patient's body so that the patient will be more comfortable and can receive over a greater length of time and/or with increased intensity.

The foregoing description and accompanying drawings set forth a number of representative embodiments at the present time. Various modifications, additions, and alternative designs will, of course, become apparent to those skilled in the art in light of the foregoing teachings without departing from the scope hereof, which is indicated by the following claims rather than by the foregoing description. All changes and variations that fall within the meaning and range of the equivalency of the claims are to be embraced within their scope.

What is claimed is:

1. A method of treating a patient via infrared radiant heating, comprising: increasing the radiant energy per unit time of infrared radiation applied to the patient's skin over a duration of a treatment protocol.

2. The method of claim 1 wherein the radiant energy per unit time is increased in a manner that the patient, after an initial warming period, does not perceive a significant temperature increase over the duration of the treatment protocol.

3. The method of claim 1 wherein the infrared radiation is applied using a source comprising one or more reflectors and each reflector has a beam guard ring to narrow the beam of radiation.

4. The method of claim 3 wherein materials for the beam guard ring are transparent or comprise a non-transparent metal.

5. The method of claim 1 wherein the infrared radiation is changed over the duration of the treatment protocol in at least one of a linearly increasing manner, an incrementally increasing manner or an incrementally increasing manner in which the energy is pulsed.

6. The method of claim 1 further comprising providing at least one speaker to provide audible information to the patient or a medical practitioner.

7. The method of claim 1 wherein radiant energy per unit time of infrared radiation applied to the patient's skin is increased to increasing plateaus during the treatment protocol.

8. The method of claim 8 wherein the duration of the treatment protocol is between 20 minutes to 3 hours.

9. The method of claim 1 wherein the treatment protocol is used to treat a condition other than a skin condition.

10. The method of claim 1 wherein the treatment protocol is used to treat a viral infection or a bacterial infection.

11. The method of claim 10 wherein the treatment protocol is used to treat fever, chills, nausea, shortness of breath or coughing.

12. The method of claim 10 wherein the treatment protocol is used to treat a Coronavirus disease, COVID-19, Severe acute respiratory syndrome (SARS), a common cold, an influenza, chickenpox, cold sores, rabies, Ebola, AIDS (HIV), or an avian influenza.

13. The method of claim 10 wherein the patient is also treated with at least one therapeutic agent.

14. The method of claim 13 wherein the therapeutic agent comprises an antibiotic, an antiviral, a pain killer, an anti-inflammatory or a health supplement such as a vitamin, a mineral, a fiber, a fatty acids or an amino acid.

15. The method of claim 1 wherein the treatment targets one or more lymph nodes.

16. The method of claim 1 wherein the treatment targets one or more of lymph nodes in the neck, lymph nodes under the shoulders, or pelvic lymph nodes.

17. A system for treating a patient, comprising:
a source for applying infrared energy to the patient for infrared radiant heating, and
a control system in operative connection with the source, the control system being conjured to control the source to increase radiant energy per unit time of infrared radiation applied to the patient over a duration of a treatment protocol.

18. The system of claim 17 wherein the control system is configured so that the radiant energy per unit time is increased in a manner that the patient, after an initial warming period, does not perceive a significant temperature increase over the duration of the treatment protocol.
